(19) United States
(12) Patent Application Publication
(10) Pub. No.: US 2025/0277190 A1
(43) Pub. Date: Sep. 4, 2025

(54) DUAL-VIAL SYSTEM AND METHOD FOR PRESERVATION OF SEMEN SAMPLES IN MAIL-IN SEMEN ANALYSIS KITS

(71) Applicants: Ashwin Ramachandran, Mel Bourne VIC (AU); Syed Basim Saroosh Zaidi, Granville NSW (AU)

(72) Inventors: Ashwin Ramachandran, Mel Bourne VIC (AU); Syed Basim Saroosh Zaidi, Granville NSW (AU)

(21) Appl. No.: 18/593,975

(22) Filed: Mar. 3, 2024

(30) Foreign Application Priority Data
Mar. 1, 2024 (GB) GB2402972.0

(51) Int. Cl. C12N 5/00

(52) U.S. Cl. CPC C12N 5/526; C12N 5/548; C12N 5/562

(57) ABSTRACT

A dual-vial system, kit, and method for preservation of semen samples in mail-in semen analysis kits are described. The system comprises a dual-vial kit that comprises a first vial for collecting a semen sample of a subject, a second vial having a predetermined volume of a stabilization medium, and a transfer device for transferring a predetermined portion of the semen sample from the first vial to the second vial such that the transfer device has a volume allowing transfer of 1 part of the semen sample to be diluted in 3 parts of the stabilization medium in the second vial. Accordingly, the dual-vial kit enables preservation of semen viability for an extended period of up to 72 hours and carrying out of semen analysis within up to 72 hours of storing the semen in the stabilization medium.

### FIELD OF THE DISCLOSURE

The present disclosure generally relates to collection, storage, and analysis of biological fluids. In particular, the present disclosure relates to mail-in sperm analysis test kits and method of usage of the kits

### BACKGROUND

Sperm analysis plays a pivotal role in male fertility diagnostics, offering crucial insights into reproductive health. However, the inconsistency in ejaculate volume poses a substantial challenge to the accuracy and reliability of this diagnostic process. Ejaculate volume varies significantly among individuals, ranging from 1.25 to 5 milliliters, with a median volume of 3.7 milliliters, as defined by the international society for sexual medicine and the world health organization (WHO). This variation presents substantial obstacles to standardizing sperm analysis, particularly in the context of at-home or mail-in diagnostic kits. Furthermore, the time-sensitive nature of sperm viability exacerbates this problem. Once semen is outside the male body, sperm rapidly loses its motility and can die within minutes, becoming rarely viable for testing after just one hour. This presents logistical challenges in handling and transporting semen samples, particularly when immediate access to a laboratory for testing is not feasible.

Existing approaches often rely on in vitro fertilization (IVF) culture media or cryopreservants as a transportation or stabilization medium. These solutions require end-users to combine the transportation medium with the semen sample in a single vial, with the aim of preserving sperm viability during transportation. However, such existing approaches introduce a critical issue known as dilution concentration variability. Dilution is not merely a matter of mathematical ratios; it extends to the biophysical properties of the semen sample. When individuals with varying ejaculate volumes, for instance, 2 milliliters and 7 milliliters, mix their samples with a fixed volume of 12 milliliters of the transportation medium, the resulting dilution ratios differ significantly. This inconsistency in dilution can impact sperm concentration and motility readings, potentially leading to inaccuracies in the diagnostic process.

Efforts have been made to address the standardization challenge by employing centrifugation techniques upon receiving samples at the laboratory. However, centrifugation, while separating sperm from other components, fails to reverse the potential damages or inaccuracies introduced by the initial improper dilution or an inadequate amount of the transportation medium. This process adds complexity and cost to the diagnostic process and is less suitable for at-home or mail-in diagnostic kits.

Existing solutions suffer from one or more limitations as described above. Therefore, there remains a need for a testing kit that preserves the quality of a semen sample for subsequent testing in a convenient way for long durations.

### OBJECTIVES OF THE DISCLOSURE

An object of the present disclosure is to provide an improved technical solution that overcomes the problems encountered in the prior art in providing a testing kit for preserving a quality of a semen sample for long durations.

Another object of the present disclosure is to provide an at-home male fertility diagnostic kit or a mail-in male fertility diagnostic kit for collecting and preserving a quality of a semen sample for subsequent testing in a convenient way for long durations.

Yet another object of the present disclosure is to provide a dual-vial system and method for preservation of semen samples in semen analysis kits.

Yet another object of the present disclosure is to describe a standardized procedure for maintaining a consistent dilution concentration of a semen sample and its corresponding transportation medium in an at-home male fertility diagnostic kit.

Yet another object of the present disclosure is to provide a procedure that allows accurate preparation of semen samples for diagnostic analysis.

Yet another object of the present disclosure is to maintain a dilution ratio of a semen sample and a transportation medium or a stabilization medium to enhance accuracy in semen sample testing.

Yet another object of the present disclosure is to facilitate various types of diagnostic analyses while maintaining sample integrity, allowing both an original semen sample and a homogenized mixture to be included in a return package for laboratory testing.

### SUMMARY OF THE DISCLOSURE

In order to achieve aforementioned objectives of the present disclosure, in one aspect, a method for collecting and preserving semen samples of a subject using a dual-vial kit, for extended temporal viability, is provided. The method comprises followings steps of collecting a neat semen sample of the subject in a first vial. transferring a predetermined portion of the semen sample from the first vial to a second vial to form a homogenized solution, the second vial has a predefined volume of a stabilization medium, and storing the homogenized solution, thereby enabling carrying out of semen analysis within up to a predefined time of storing the semen sample in the stabilization medium and enabling preservation of semen viability for an extended period of up to the predefined time. The predefined time may be 72 hours.

In some embodiments, the method further comprises agitating a mixture of the predetermined portion of the semen sample and the stabilization medium till the homogenized solution is formed, after transferring the predetermined portion of the semen sample from the first vial to the second vial.

In some embodiments, a step of transferring the predetermined portion of the semen sample from the first vial to the second vial further comprises mixing the predetermined portion of the semen sample in the stabilization medium in a dilution ratio of 3:1 to form a mixture. The mixture has 3 parts of the stabilization medium and 1 part of the semen sample.

In some embodiments, the method further comprises storing the homogenized solution in the second vial at near body temperature. In one embodiment, the method further comprises storing the homogenized solution in the second vial at temperatures between 36.1 Celsius (° C.) and 37.2° C.

In some embodiments, the method further comprises maintaining or storing the stabilization medium at temperatures between 2 Celsius (° C.) and 12 (° C.), prior to mixing with the semen sample.

In one embodiment, the stabilization medium comprises one or more ingredients comprising one or more pH buffer agents, one or more inorganic salts, one or more organic compounds and one or more amino acids with antioxidant properties. In some embodiments, the one or more ingredients of the stabilization medium are dissolved in water to formulate the stabilization medium.

In some embodiments, the method further comprises storing a remaining semen sample in the first vial as a backup for transporting along with the second vial comprising the homogenized solution.

Optionally, the predetermined portion of the semen sample from the first vial is transferred to the second vial using a transfer device. Optionally, the transfer device is a syringe.

In another aspect, a system for collecting and preserving semen samples of a subject for extended temporal viability is provided. The system comprises a dual-vial kit. The dual-vial kit comprises a first vial for collecting a semen sample of the subject, a second vial having a predetermined volume of a stabilization medium, and a transfer device for transferring a predetermined portion of the semen sample from the first vial to the second vial such that the transfer device has a volume that allows transfer of 1 part of the semen sample to be diluted in 3 parts of the stabilization medium in the second vial.

In yet another aspect, a dual-vial kit for collecting, packaging, storing, and transporting semen samples of a subject is provided. The dual-vial kit comprises a first vial for collecting a semen sample of the subject, a second vial having a predetermined volume of a stabilization medium, and a transfer device for transferring a predetermined portion of the semen sample from the first vial to the second vial such that the transfer device has a volume that allows transfer of 1 part of the semen sample to be diluted in 3 parts of the stabilization medium in the second vial.

In some embodiments, the first vial and the second vial are configured to be sealed to ensure an airtight and leak-proof seal to maintain integrity of contents.

In some embodiments, the dual-vial kit further comprises labelling for the first vial and the second vial.

In some embodiments, the dual-vial kit further comprises a protective outer packaging configured to house the first vial, the second vial, and the transfer device. Optionally, the transfer device is a syringe.

In some embodiments, the dual-vial kit further comprises an instruction manual for collecting, packaging, storing, and transporting the semen sample of the subject.

The system, kit and method of the present disclosure is advantageous that enable maintaining a consistent dilution concentration of a semen sample and its corresponding transportation medium in an at-home male fertility diagnostic kit or mail-in male fertility diagnostic kit. Further, the kit and method of the present disclosure enables storage of semen samples up to 72 hours. Accordingly, the kit and method of the present disclosure allows for accurate preparation of semen samples for various types of diagnostic analyses while maintaining sample integrity which in turn improves accuracy in semen sample testing. Further, the kit and method of the present disclosure allows both an original semen sample and a homogenized mixture of semen sample and its corresponding transportation medium to be included in a return package for laboratory testing.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The essence and intricacies of the current invention can be further elucidated through a detailed examination of the accompanying figures, which serve to illuminate the various objectives, features, components, and advantages to those who possess expertise in this field. These figures, when considered in conjunction with the text of this patent application, provide a holistic understanding of the invention's aims, functionalities, and advantages. They serve as a valuable resource for translating the invention's technical aspects into actionable steps that can be effortlessly understood and implemented by professionals in the field.

FIG. 1 is an exemplary block diagram of a system for collecting, packaging, storing, and transporting semen samples of a subject in accordance with the present disclosure.

FIG. 2 is an exemplary schematic illustration of a dual-vial kit in accordance with the present disclosure.

FIG. 3 is a flow chart that illustrates a method for collecting and preserving semen samples of a subject using a dual-vial kit in accordance with the present disclosure.

### DETAILED DESCRIPTION

The following description describes various features and functions of the disclosed system, kit, and method with reference to the accompanying figures. The terminology used herein is for the purpose of describing particular embodiments only and is not intended to limit the scope of the present invention.

The detailed description is construed as a description of the currently preferred embodiments of the present invention and does not represent the only form in which the present invention may be practiced. This is to be understood that the same or equivalent functions may be accomplished, in any order unless expressly and necessarily limited to a particular order, by different embodiments that are intended to be encompassed within the scope of the present invention.

The embodiment is chosen and described to provide the best illustration of the principles of the invention and its practical application and to enable one of ordinary skill in the art to utilize the invention in various embodiments and with various modifications as are suited to the particular use contemplated.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

As mentioned, there remains a need for an improved technical solution that overcomes a problem of inconsistency in dilution of a semen sample in a stabilization medium due variable semen volume. The present disclosure provides systems, kits, and method for collecting, packaging, storing, and transporting semen samples of a subject. The kit and method of the present disclosure is advantageous that enable maintaining a consistent dilution concentration of a semen sample and its corresponding transportation medium in an at-home male fertility diagnostic kit or a mail-in male fertility diagnostic kit. Further, the kit and method of the present disclosure enables storage of semen samples up to 72 hours. Accordingly, the kit and method of the present disclosure allows for accurate preparation of semen samples for various types of diagnostic analyses while maintaining sample integrity which in turn improves accuracy in semen sample testing. Further, the kit and method of the present disclosure allows both an original semen sample and a homogenized mixture of semen sample and its corresponding transportation medium to be included in a return package for laboratory testing.

As used herein, several terms are defined below:

The term “semen sample” as used herein, generally refers to an organic bodily fluid secreted by the male gonads and other sexual organs of male or hermaphroditic animals and can fertilize the female ovum.

The term “subject,” as used herein, generally refers to a biological entity containing expressed genetic materials. The biological entity may be a mammal. The mammal may be a human. In some embodiments, the mammal may be a non-human mammal. The non-human mammal may be a genetically modified non-human mammal or a naturally occurring non-human mammal. In some embodiments, the biological entity may be a non-mammal.

The term “semen analysis” as used herein, generally refers to is a laboratory test that is performed to assess male fertility.

The term “neat semen sample” as used herein, generally refers to a clean and hygiene semen sample. stabilization medium

The terms “stabilization medium” and “transportation medium” are used interchangeable herein. They refer to a substance or a solution or a material designed to maintain the integrity, viability, or stability of biological specimens or materials during storage or transportation. It is used to prevent degradation, damage, or alteration of the biological components, ensuring that they remain in a suitable condition for subsequent analysis or processing. The biological specimens or materials may be a semen sample.

The term “temporal viability” as used herein, generally refers to an ability of a biological material to remain alive, functional, or viable over a specific period of time. It is a measure of how well an organism, cell, or biological material can maintain its integrity and functionality over a given temporal duration.

The term “extended temporal viability” as used herein, generally refers to an ability of a biological material to remain alive, functional, or viable over a prolonged period of time.

The term “vial” as used herein, generally refers to a container often used to store and transport liquids, powders, or other substances or materials. Vials come in various sizes and materials. The vial may be provided with a stopper or cap to seal and provide secure environment to protect the contents from contamination and external factors.

The term “first vial” as used herein, generally refers to a container to be used to collect the neat semen sample in the context of the present disclosure.

The term “second vial” as used herein, generally refers to a container to be used to store the stabilization medium before mixing with the semen sample and to store a homogenized solution comprising the stabilization medium and the semen sample after pouring the semen sample to the second vial.

The term “kit” as used herein, generally refers to a set of items, materials, or components packaged together for a specific purpose.

The term “dual-vial kit” as used herein, generally refers to a kit encompassing two vials in addition to other components or items for a specific purpose or function.

The terms “predetermined volume” “predetermined portion” “predefined volume” as used herein, generally refers to a specific and known amount of a substance or a solution or a material set in advance for a particular purpose, ensuring accuracy and reproducibility in various scientific, industrial, or analytical applications.

The term “transfer device” as used herein, generally refers to an instrument or apparatus such as a pipette, a syringe, or a similar instrument, for transferring a substance from one container to another container with precision.

The term “labelling” as used herein, generally refers to an identifier such as a tag, or a sticker, attached or affixed on vials or other products of a kit. The purpose of labeling is to provide essential details, instructions, warnings, or identification, making it easier to understand the nature of the item or its contents.

The term “protective outer packaging” as used herein, generally refers to an external packaging structure specifically designed to accommodate and components or products of a kit.

The term “preservation” as used herein, generally refers to an act or process of maintaining the quality, condition, or existence of a substance or a solution or a material over time.

The term “storing” as used herein, generally refers to an act or process of keeping, placing, or maintaining items or substances or solution or material in a designated location for a specific period. The goal of storing is to preserve the items or substances or solution or material.

The term “collection” as used herein, generally refers to an act or process of gathering or accumulating specimens or representative portions of a material, substance, or substance mixture or solution for further analysis, examination, or study.

The term “packaging” as used herein, generally refers to an act or process of enclosing a substance or a solution or a material in a container or vial to protect them.

The term “transporting” as used herein, generally refers to an act or process of movement or conveyance of collected specimens, materials, or substances from one location to another location.

The term “dilution ratio” as used herein, generally refers to a proportional relationship between the amount of a first substance or material or solution and the amount of second substance or material or solution used to create a solution of a desired concentration.

The term “agitating” as used herein, generally refers to an act or process of stirring, or shaking a substance or mixture vigorously to promote mixing, dispersion, or homogenization.

The term “homogenized solution” as used herein, generally refers to a mixture in which the components are uniformly distributed.

The term “semen viability” as used herein, generally defined as a percentage of live sperm found in a semen sample.

The term “near body temperature” as used herein, generally refers to a temperature that is close to the average internal temperature of the human body.

The term “pH buffer agent” as used herein, generally refers to a substance or a mixture of substances that helps to maintain a relatively constant pH (acidity or alkalinity) in a solution, even when an acid or a base is added.

The term “inorganic salts” as used herein, generally refers to a chemical compound consisting of an ionic assembly of positively charged cations and negatively charged anions, which results in a compound with no net electric charge.

The term “organic compound” as used herein, generally refers to a chemical compound in which one or more atoms of carbon are covalently linked to atoms of other elements.

The term “amino acids with antioxidant properties” as used herein, generally refers to amino acids that possess the ability to neutralize or reduce oxidative stress and prevent the damaging effects of free radicals within a biological or chemical system.

The term “one or more” as used herein, generally refers to at least one and also more than one.

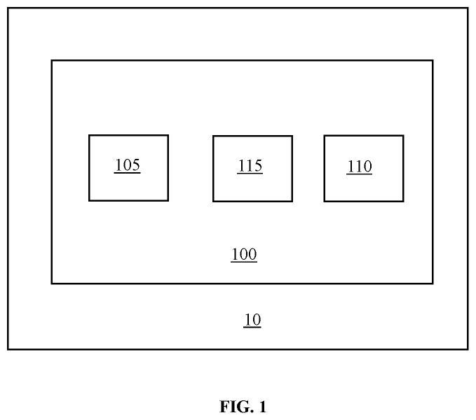

Referring now to the drawings, and more particularly to FIGS. 1 through 3, where similar reference characters denote corresponding features consistently throughout the figures, example embodiments are shown.

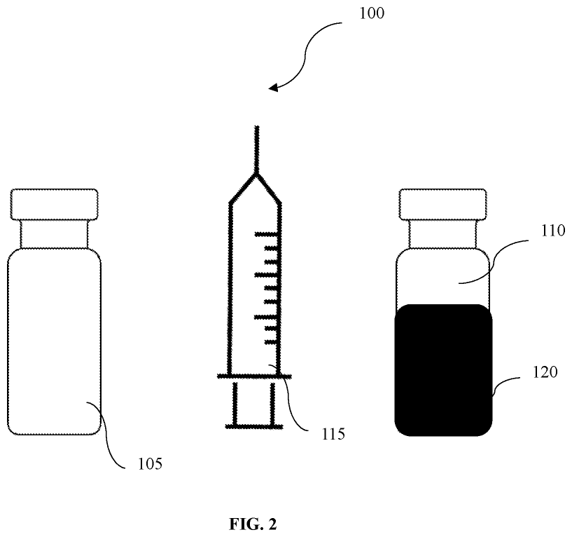

FIG. 1 is an exemplary block diagram of a system 10 for collecting, packaging, storing, and transporting semen samples of a subject in accordance with the present disclosure. The system 10 comprises a dual kit 100 for collecting, packaging, storing, and transporting the semen samples of the subject. In one exemplary embodiment, the subject is a human. The semen sample may be a human semen sample. The system 10 may further comprise a diagnosis kit for analyzing the semen sample to assess male fertility using methods known to those of skill in the art. FIG. 2 is an exemplary schematic illustration of the dual-vial kit 100 in accordance with the present disclosure. The dual-vial kit 100 is a specialized collection, packaging, and transport solution for semen samples. In one exemplary embodiment, the dual-vial kit 100 is used for collecting, packaging, and transporting human semen samples. The dual-vial kit 100 has two separate vials namely a first vial 105 and a second vial 110, sealed with a specialized cap or rubber stopper to ensure an airtight and leak-proof seal to maintain the integrity of the contents. The first vial 105 and the second vial 110 are sterile and transparent. It is to be noted that the first vial 105 and the second vial 110 are made of any suitable material that is chemically resistant with the semen sample and/or the stabilization medium 120. Examples of materials used for making the first vial 105 and the second vial 110 includes polypropylene, polystyrene, polyethylene, and borosilicate glass. In some embodiments, the first vial 105 and the second vial 110 are made from materials specifically designed for low to high temperature storage. The first vial 105 and the second vial 110 may have capacities ranging from a few milliliters to several milliliters. The first vial 105 and the second vial 110 may be in same size. In some embodiments, the first vial 105 and the second vial 110 may be in different size. The first vial 105 and the second vial 110 may comprise a graduation mark for indicating an amount and/or level of the semen sample in the vials. Usage of two vials helps in maintaining integrity of the semen sample mixed in the stabilization medium 120 in the second vial 110 and neat human semen as a backup in the first vial 105. The second vial 110 is shown to have a predetermined volume of the stabilization medium 120. The dual-vial kit 100 further has a transfer device 115 to facilitate the transfer of the semen sample from the first vial 105 to the second vial 110. The transfer device 115 may come in various forms, such as a spike, needle, or specialized connector, depending on the specific application. In some embodiments, the transfer device 115 is a syringe. It is to be noted that the transfer device 115 is sterile. The transfer device 115 ensures a controlled and accurate transfer of the semen sample from one vial to another. Similar to vials, the transfer device 115 may comprise a graduation mark for indicating an amount and/or level of the semen sample for enabling controlled and accurate transfer of the semen sample from one vial to another. The transfer device 115 is made of any suitable material that is chemically resistant with the semen sample. Examples of materials used for making the transfer device 115 includes polypropylene, polystyrene, polyethylene, and borosilicate glass.

In an embodiment of the present disclosure, the dual-vial kit 100 has provision of clear and informative labelling on the vials and packaging. The labelling may provide essential information such as identification of a user, names of substances, and any necessary warnings. In another embodiment of the present disclosure, the dual-vial kit 100 has an instruction manual to guide healthcare professionals through the proper process of preparation of kit before sample collection, sample collection, storage, and transportation. The dual-vial kit 100 has a protective housing to house in a protective outer packaging, which may be a box, blister pack, or another form of container. The protective outer packaging may be made of any suitable material that protects the vials and the transfer device 115 from contamination and external factors such as physical damage, temperature variations, and exposure to light or moisture. The protective outer packaging acts as a barrier against contaminants, maintains a sterile environment, and prevents the ingress of dust, microorganisms, or other impurities. The protective outer packaging is durable, resistant to tearing or puncturing, and capable of withstanding the rigors of transportation. In some embodiments, the dual-vial kit 100 comprises temperature regulating elements to maintain the temperature of the first vial 105 and the second vial 110 before and after use of the dual-vial kit 100. In some embodiments, the temperature regulating elements includes, but not limited to, ice packs, gel packs, and heat packs. In some embodiments, other temperature regulating elements such as films or laminates are used or applied on the exterior of a cardboard box which houses the dual vial kit 100 for maintaining temperature of the stabilization medium 120, for example. In some embodiments, one or more compartments in the protective outer packaging built-in with temperature control mechanisms to actively regulate the temperature of the first vial 105 and the second vial 110 after receiving the vials in the protective outer packaging. For example, the temperature regulating elements/mechanisms maintains temperature of the second via 110 between 2 degrees Celsius (° C.) and 12° C. when the second via 110 comprising the stabilization medium 120 only. In another example, the temperature regulating elements/mechanisms maintains temperature of the second vial 110 to near body temperature when the second vial 110 comprising a homogenized solution of the stabilization medium 120 and the semen sample. In some embodiments, the dual-vial kit 100 further comprises, but not limited to, alcohol swabs or disinfectant wipes for sterilizing the skin or equipment before sample collection, disposable gloves to ensure aseptic conditions and protect the user during sample collection or handling. In some embodiments, the dual-vial kit 100 further comprises temperature indicator to indicate temperature each vial in the dual-vial kit 100, thereby enabling to monitor the temperature of each vial.

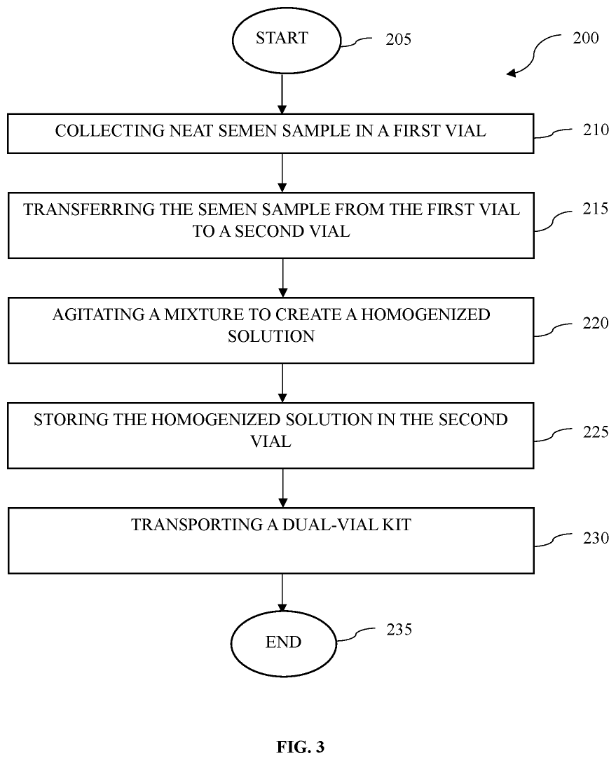

FIG. 3 shows a flow chart 200 of using the dual-vial kit 100, in accordance with present disclosure for collecting, packaging, storing, and transporting semen samples of the subject. The flow chart 200 starts at step 205. At step 210, neat semen sample is collected in the first vial 105. In one exemplary embodiment, the semen sample is human semen sample. Using the transfer device 115, a predetermined portion of the semen sample from the first vial 105 is transferred to the second vial 110, at step 215. The second vial 110 has a predefined volume of a stabilization medium 120 in it. The predetermined portion of the semen is transferred such that a dilution ratio of 3:1 is maintained (i.e., three parts of the stabilization medium and one part semen). This ensures the semen's stability and viability for up to 72 hours for subsequent analysis. In one exemplary embodiment, 1 ml of semen sample is added to the 3 ml of the stabilization medium 120.

In some embodiments of the present disclosure, the second vial 110 is prepared with

predetermined volume of the stabilization medium 120. The stabilization medium 120 has one or more ingredients comprising one or more pH buffer agents, inorganic salts, organic compounds, and amino acids with antioxidant properties. The one or more ingredients of the stabilization medium 120 are dissolved in water. The second vial 110 may be prepared in advance or before collecting the semen sample. In an embodiment, when the second vial 110 prepared in advance, the second vial 110 is maintained at temperatures between 2° C. and 12° C. In some embodiments, the second vial 110 comprising the stabilization medium 120, is stored between 2° C. and 12° C. using ice packs, and so forth. In some embodiments, microprocessor-controlled cooling devices may be used for maintaining temperature of the second vial 110 comprising the stabilization medium 120 between 2° C. and 12° C. It is to be noted that when the stabilization medium 120 is not in use and has not been mixed with the semen sample, the stabilization medium 120 is stored between 2° C. and 12° C., through a use of a fridge, or in the case of the dual-vial kit 100 being transported, through the use of a gel ice pack, any other cooling elements or devices and the like. Before the stabilization medium 120 is mixed with the semen sample, the stabilization medium 120 is brought to room temperature, thereafter the stabilization medium 120 is mixed with the semen sample. The mixture comprising the stabilization medium 120 and the semen sample is to be maintained near body temperature and not to be cooled after it is mixed together. In some embodiments, the pH buffer agents may be selected from a group comprising of HEPES (4-(2-hydroxyethyl)-1-piperazineethanesulfonic acid), TES (N-tris(hydroxymethyl)methyl-2-aminoethanesulfonic acid), Tris (Tris(hydroxymethyl)aminomethane), MOPS (3-(N-morpholino)propanesulfonic acid), and phosphate buffer. In some embodiments, the inorganic salts may be selected from a group comprising of sodium chloride (NaCl), potassium chloride (KCl), calcium chloride (CaCl₂), sodium bicarbonate (NaHCO₃), magnesium sulfate (MgSO₄), and sodium citrate. In some embodiments, the organic compounds may be selected from a group comprising of proteins, sugar, osmolytes, chelating agents, lipids, and enzymes. In some embodiments, the amino acids with antioxidant properties may be selected from a group comprising of Alanine, Aspartate, Asparagine, Glutamate,

Alanyl-Glutamine, Glycine, Proline, and Serine.

Subsequently at step 220, the mixture of semen sample and the stabilization medium 120 is agitated to create a homogenized solution. The specifics of the required agitation, such as the duration and intensity, may vary but should be sufficient to ensure uniform mixing.

Thereafter, the step 225, the homogenized solution in the second vial 110 is stored at near body temperature. In an embodiment of the present disclosure, the second vial 110 is stored at temperature between 36.1° C. and 37.2° C. In an embodiment of the present invention, the first vial 105 with the remaining human semen is also stored as a backup. In some embodiments, the dual-vial kit 100 includes any of the following for maintaining temperature of the homogenized solution in the second vial 110: foam inserts or liners, phase change materials, microprocessor-controlled heating devices, insulating wraps or films or laminates, and gel packs.

Finally, at step 230 the dual-vial kit 100 is transported for storage or analysis. The stored homogenized solution in the second vial 110 can be used for analysis of the semen sample within up to 72 hours from the time it was initially mixed with the stabilization medium 120, allowing for the extended preservation of semen viability. The flowchart 200 end at step 235.

The dual-vial kit 100 and method for preserving semen samples using the dual-vial kit 100 kit offer several significant advantages in at-home male fertility diagnostics. By employing a 3:1 dilution ratio of semen to the stabilization medium 120 and providing a neat control sample in the first vial 105, the present invention ensures both the preservation of sample integrity and the capacity for a broad spectrum of diagnostic analyses. The present disclosure also solves the problem associated with the variable semen volume, helping with accurate analysis. Further, the present disclosure allows the usage preserving the quality of human semen sample for up to 72 hours.

It is to be understood that while we have illustrated and described certain forms of the present invention, it is not to be limited to the specific forms or arrangement of parts herein described and shown.

It will be apparent to those skilled in the art that various changes may be made without departing from the scope of the invention and the invention is not to be considered limited to what is shown in the drawings and described in the specification. Several features are described hereinafter that can each be used independently of one another or with any combination of other features. However, an individual feature might only address one of the problems discussed above. Some of the problems discussed above might not be fully addressed by any of the features described herein.

### LIST OF REFERENCE NUMERALS

10—system

100—dual-vial kit

105—first vial

110—second vial

115—transfer device

120—stabilization medium

What is claimed is:

1. A method for collecting and preserving semen samples of a subject, using a dual-vial kit, for extended temporal viability, the method comprising:
collecting a neat semen sample of the subject in a first vial;
transferring a predetermined portion of the semen sample from the first vial to a second vial to form a homogenized solution, wherein the second vial has a predefined volume of a stabilization medium; and
storing the homogenized solution, thereby enabling carrying out of semen analysis within up to a predefined time of storing the semen sample in the stabilization medium and enabling preservation of semen viability for an extended period of up to the predefined time.

2. The method of claim 1, further comprising agitating a mixture of the predetermined portion of the semen sample and the stabilization medium till the homogenized solution is formed, after transferring the predetermined portion of the semen sample from the first vial to the second vial.

3. The method of claim 1, wherein transferring the predetermined portion of the semen sample from the first vial to the second vial further comprises mixing the predetermined portion of the semen sample in the stabilization medium in a dilution ratio of 3:1 to form a mixture, wherein the mixture has 3 parts of the stabilization medium and 1 part of the semen sample.

4. The method of claim 1, further comprising storing the homogenized solution in the second vial at near body temperature.

5. The method of claim 1, further comprising storing the homogenized solution in the second vial at temperatures between 36.1 Celsius (° C.) and 37.2° C.

6. The method of claim 1, further comprising maintaining or storing the stabilization medium at temperatures between 2 Celsius (° C.) and 12 (° C.), prior to mixing with the semen sample.

7. The method of claim 1, wherein the stabilization medium comprises one or more ingredients comprising one or more pH buffer agents, one or more inorganic salts, one or more organic compounds and one or more amino acids with antioxidant properties, wherein the one or more ingredients of the stabilization medium are dissolved in water.

8. The method of claim 1, further comprising storing a remaining semen sample in the first vial as a backup for transporting along with the second vial comprising the homogenized solution.

9. The method of claim 1, wherein the predetermined portion of the semen sample from the first vial is transferred to the second vial using a transfer device, wherein the transfer device is a syringe.

10. The method of claim 1, wherein the predefined time is 72 hours.

11. A system for collecting and preserving semen samples of a subject for extended temporal viability, the system comprising:
a dual-vial kit that comprises
a first vial for collecting a semen sample of the subject;
a second vial having a predetermined volume of a stabilization medium; and
a transfer device for transferring a predetermined portion of the semen sample from the first vial to the second vial such that the transfer device has a volume that allows transfer of 1 part of the semen sample to be diluted in 3 parts of the stabilization medium in the second vial.

12. A dual-vial kit for collecting, packaging, storing, and transporting semen samples of a subject, the dual-vial kit comprising:
a first vial for collecting a semen sample of the subject;
a second vial having a predetermined volume of a stabilization medium; and
a transfer device for transferring a predetermined portion of the semen sample from the first vial to the second vial such that the transfer device has a volume that allows transfer of 1 part of the semen sample to be diluted in 3 parts of the stabilization medium in the second vial.

13. A dual-vial kit of claim 12, wherein the first vial and the second vial are configured to be sealed to ensure an airtight and leak-proof seal to maintain integrity of contents.

14. The dual-vial kit as claimed in claim 12, further comprising labelling for the first vial and the second vial.

15. The dual-vial kit of claim 12, further comprising a protective outer packaging configured to house the first vial, the second vial, and the transfer device, wherein the transfer device is a syringe.

16. The dual-vial kit of claim 12, further comprising an instruction manual for collecting, packaging, storing, and transporting the semen sample of the subject.

17. The dual-vial kit of claim 12, wherein the stabilization medium comprises one or more ingredients comprising one or more pH buffer agents, one or more inorganic salts, one or more organic compounds and one or more amino acids with antioxidant properties, wherein the one or more ingredients of the stabilization medium are dissolved in water.
